(19) United States
(12) Patent Application Publication
(10) Pub. No.: US 2021/0290550 A1
(43) Pub. Date: Sep. 23, 2021

(54) Process for Preparing Rapidly or Very Rapidly Dissolving Tablets Comprising Freely Soluble API

(71) Applicant: Sandoz AG, Basel (CH)

(72) Inventors: Veronika Werner, Kufstein (AT); Anke Schuster, Reith im Alpbachtal (AT); Stephanie Buxbaum, Oberaudorf (DE)

(73) Assignee: Sandoz AG, Basel (CH)

(21) Appl. No.: 17/260,291

(22) PCT Filed: Jul. 25, 2019
(86) PCT No.: PCT/EP2019/070014

§ 371 Date: Jan. 14, 2021

(30) Foreign Application Priority Data
Jul. 27, 2018 (EP) 18186196.4
Feb. 27, 2019 (EP) 19159736.8

(51) Int. Cl. A61K 9/20; A61K 9/28; A61K 31/513; A61K 31/65

(52) U.S. Cl. CPC A61K 9/2077; A61K 9/2095; A61K 9/2893; A61K 31/65; A61K 9/2054; A61K 9/2013; A61K 31/513; A61K 9/28

(57) ABSTRACT

The present invention relates to a process for preparing a rapidly or very rapidly dissolving tablet comprising an at least freely soluble solid form of an active pharmaceutically ingredient (API). The tablet comprises a granulate comprising said at least freely soluble solid form of an active pharmaceutically ingredient in form of granules, and a tablet base, with the granules exhibiting a certain minimum size, and the tablet base comprising a disintegrant. The process comprises a step of mixing the API-containing granulate with the tablet base, followed by a step of compressing the mixture to form the tablet. The present invention also relates to a rapidly or very rapidly dissolving tablet comprising an at least freely soluble solid form of an API, wherein said tablet comprises a mixture of API-containing granulate comprising API-containing granules, and a disintegrant-containing tablet base, with the granules exhibiting a certain minimum size. The amount of said API in the tablet is at least 10% w/w based on the weight of the tablet.

### FIELD OF THE INVENTION

The present invention relates to a process for preparing a rapidly or very rapidly dissolving tablet comprising an at least freely soluble solid form of an active pharmaceutically ingredient (API). The tablet comprises granulate comprising said at least freely soluble solid form of the API in form of granules, and a tablet base, with the granules exhibiting a certain minimum size, and the tablet base comprising a disintegrant. The process comprises a step of mixing the API-containing granulate with the tablet base, followed by a step of compressing the mixture to form the tablet.

The present invention also relates to a rapidly or very rapidly dissolving tablet comprising an at least freely soluble solid form of an API, wherein said tablet comprises a mixture of granulate comprising API-containing granules and a disintegrant-containing tablet base, with the granules exhibiting a certain minimum size. The amount of said API in the tablet is at least 10% w/w based on the weight of the tablet.

### BACKGROUND OF THE INVENTION

Active pharmaceutical ingredients (API) for medical use exhibit highly variable and compound-specific, characteristic properties. Such a property is, for instance, the solubility of the API in different environments or media, which also depends on the particular solid form of an API used. The formulation of a pharmaceutical composition or dosage form takes these properties of the API into account, as well as further parameters, such as the route of administration, the desired site of action, or the timing of the onset of effect of the API.

In order to administer a particular API to patients, said API has to be formulated into a suitable pharmaceutical dosage form. Formulation design aims at providing pharmaceutical dosage forms comprising a desired API which formulations then show desired release properties depending on where the API is to act in the body. Significant research has gone into the formulation of APIs having low solubility in aqueous environments, while the formulation of highly soluble APIs is often thought of as being “easy”.

However, this perception is too simplistic.

Johnson et al. Journal of Pharmaceutical Sciences (1991), Vol. 80, pages 469-471 describe that highly soluble and/or hygroscopic ingredients decrease the effectiveness of super-disintegrants in promoting in vitro dissolution. They observed that highly soluble sorbitol formulations that contain super-disintegrants dissolve at a slower rate compared with less soluble lactose formulations with super-disintegrants. Johnson et al. conclude that the decrease in efficiency of the disintegrants can be addressed by careful selection of tablet excipients.

Sometimes fast release of the API at the site of action or resorption is desired. Thus there is a need for dosage forms that disintegrate rapidly and release the API quickly, for example either in the stomach or in the duodenum.

In addition to the dissolution behaviour of the pharmaceutical formulation/dosage form (and depending thereon, the absorption of the API into the circulation), a further important aspect when formulating dosage forms is the drug load, that is, the ratio of API (“drug”) to excipients being present in the dosage form. A high drug load may have the result that fewer dosage forms have to be taken by patients, e.g. the pill burden is lower, and this in turn can contribute to an improved patient compliance. For certain APIs that are needed at higher concentrations for the therapeutic effect, a high drug load becomes particularly important if one wants to avoid an unnecessary pill burden for the patient.

There is therefore a need for a reliable formulation process that provides pharmaceutical dosage forms having a high drug load of an at least freely soluble API and yet disintegrate quickly at the desired site of API release.

### SUMMARY OF THE INVENTION

The present inventors have found that pharmaceutical dosage forms having a high drug load of an at least freely soluble API can have problems with disintegration and thus API release. Tablets containing at least 5% elagolix, for example, did not qualify as rapidly dissolving or very rapidly dissolving, even though they contained sufficient amounts of disintegrant.

The present inventors have then discovered that by applying the process of the present invention, the disintegration properties of a tablet comprising a relatively high drug (API) load of an at least freely soluble solid form of an API can be improved.

The process of the present invention aims at achieving a certain degree of spatial separation between disintegrant and the at least freely soluble solid form of the API. This spatial separation affects properties like for instance the disintegration behavior of the tablet and thus the release of said API from said tablet, and/or the dissolution profile of said API in said tablet.

It has been found by the inventors that such advantageous properties can be obtained when, by applying the inventive process, a tablet is prepared from a tablet base and—embedded therein—granulate comprising granules exhibiting a certain minimum size and containing said at least freely soluble solid form of an API. It has further been found that the presence of a certain minimum amount of said at least freely soluble solid form of the API in the granules or in the granulate, based on the total weight of the granules or of the granulate, can additionally contribute to the advantageous properties.

The tablet base comprises one or more disintegrants. Preferably the tablet base should contain most of the disintegrant which is present in the tablet, while the granulate should contain most of the API which is present in the tablet. In other words, a certain spatial separation between the disintegrant and said API contributes to the at least satisfactory disintegration and/or dissolution properties of the tablet. The advantageous properties can additionally be enhanced if the granules or the granulate comprise(s) a certain minimum amount of said at least freely soluble API, based on the total weight of the granules or of the granulate.

Without wishing to be bound by any theory, it is believed that this spatial separation of the disintegrant and the at least freely soluble solid form of an API significantly decreases the competition for water between the disintegrant and said API at the stage of tablet disintegration in an aqueous environment of the human body. In particular, the presence of the disintegrant(s) (or at least a sufficient part thereof) in the tablet base provides that the disintegrant(s) come into contact with water first, which results in disintegration of the dosage form (such as a tablet) and the release of the granulate containing said API contained therein, respectively. The consequence is an improved promotion of the breakup of the dosage form into smaller fragments in said aqueous environment, thereby increasing the available surface area and thus contributing to a more rapid release of the at least freely soluble API.

Accordingly, the present invention provides the following aspects, subject-matter and preferred embodiments which, respectively taken alone or in combination, contribute to providing improved technical effects and to solving the afore-mentioned object of the invention:

1. Process for the preparation of a rapidly or very rapidly dissolving tablet comprising a very soluble or freely soluble solid form of an active pharmaceutically ingredient (API), wherein the tablet comprises a granulate and a tablet base, wherein the process comprises the following steps:
a) preparing said granulate comprising said very soluble or freely soluble solid form of an API in form of granules, wherein at least 90% w/w of said granules have a size of at least 150 μm;
b) mixing the granulate obtained in step a) with said tablet base comprising one or more disintegrants and optionally further excipients;
c) compressing the mixture from step b) to form said tablet,
wherein said granulate comprises at least 90% w/w of the total amount of said very soluble or freely soluble solid form of the API being present in the tablet,
and wherein the amount of said very soluble or freely soluble solid form of said API in the tablet is at least 10% w/w based on the weight of the tablet, and
wherein the tablet base comprises one or more disintegrants.
2. Process according to item 1, wherein the amount of said very soluble or freely soluble solid form of an API in said granulate is at least 50% w/w, based on the total weight of said granulate; preferably the amount of said very soluble or freely soluble solid form of an API in said granulate is at least 60% w/w, or at least 70% w/w; more preferably at least 80% w/w, at least 90% w/w or at least 95% w/w; respectively based on the total weight of said granulate; and most preferably, said granulate essentially consist of said very soluble or freely soluble solid form of an API.
3. Process according to item 1 and 2, wherein at least 95% w/w of said granules, preferably at least 97% w/w of said granules, and most preferred all of said granules have a size of at least 150 μm.
4. The process according to any of the preceding items, wherein
a) the solid form of the API is very soluble or freely soluble in FASSIF-medium according to European Pharmacopoeia 9.4, Section 5.11, Character Section in Monograph; or
b) the solid form of the API is very soluble or freely soluble in 0.1N HCl-solution according to European Pharmacopoeia 9.4, Section 5.11, Character Section in Monograph.
5. The process according to any of the preceding items, wherein the granules of step (a) have a size of at least 200 μm, preferably at least 300 μm, more preferably at least 400 μm, and further preferred at least 500 μm, wherein the size is preferably at most 1500 μm or at most 1200 μm, for example wherein the granule size is in a range from 200 μm to 1500 μm or to 1200 μm, or from 300 μm to 1500 μm or 1200 μm, or from 400 μm to 1000 μm, preferably in a range from 500 μm to 1500 μm.

In a further preferred embodiment, the granules of step (a) have a size of at least 150 μm, preferably of at least 250 μm, and more preferably of at least 500 μm. Examples of preferred ranges are from 150 μm to 250 μm, more preferably from more than 250 μm to 500 μm. Even more preferably, the size of the granules of step (a) is in a range from 500 μm to 1500 μm, or more than 500 μm to 1500 μm.

In a further preferred embodiment, the granules of step (a) have a size of at least 150 μm, preferably of at least 250 μm. It is additionally further preferred that the upper limit of the size of said granules is respectively 500 μm. It is for example preferred that the size of the granules of step (a) is in a range from 150 μm to 500 μm, preferably in a range from 250 μm to 500 μm.

6. The process according to any preceding item, wherein the tablet obtained after compression comprises granules comprising said very soluble or freely soluble solid form of an active pharmaceutically ingredient (API) wherein, when the tablet is broken in half by hand and the surface of the break is analysed by optical microscopy, at least five of said granules have an apparent surface area of at least 30000 μm², such as from 50000 μm² to 200000 μm².
7. The process according to any preceding item, wherein the amount of said very soluble or freely soluble solid form of said API in the tablet is at least 15% w/w, such as at least 20% w/w, at least 25% w/w, at least 30% w/w, or at least 35% w/w, based on the weight of the tablet.
8. The process according to any preceding item, wherein the amount of said very soluble or freely soluble solid form of said API in the tablet is from 15% w/w to 60% w/w, such as from 15% w/w to 50% w/w, such as from 15% w/w to 40% w/w, for example from 20% w/w to 60% w/w, from 20% w/w to 50% w/w, such as from 20% w/w to 40% w/w, based on the weight of the tablet.
9. The process according to any preceding item, wherein the tablet comprises at least one further solid form of an API, wherein said further solid form of an API is intragranular when the further solid form of an API is a very soluble or freely soluble solid form.
10. The process according to any preceding item, wherein in step (a) the granulate is prepared by a dry or wet granulation process.
11. The process according to any preceding item, wherein the granulate is prepared by a dry granulation process, such as by a slugging technique or by using a compactor, for example a roller compactor, preferably wherein the granulate is prepared by a roller compactor technique.
12. The process according to any preceding item, wherein the granulate of step (a) comprise at most 10%, such as at most 7%, or at most 5%, or at most 2.5% w/w disintegrants, based on the total amount of disintegrant being present in the tablet, preferably wherein the granulate of step (a) comprises no disintegrant.
13. The process according to any preceding item, wherein the granulate of step (a) comprise at most 10% w/w, such as at most 7% w/w, or at most 5% w/w, or at most 2.5% w/w disintegrant(s), based on the total weight of the granulate.
14. The process according to any of the preceding items, wherein the amount of said very soluble or freely soluble solid form of said API in the granulate of step a) is at least 90% w/w, preferably at least 93% w/w, more preferably at least 95% w/w, such as 97.5% w/w, and most preferably 100% w/w, based on the total amount of API being present in the tablet.
15. The process according to any of the preceding items, wherein the tablet base comprises at least 90% w/w, preferably at least 93% w/w, more preferably at least 95% w/w, such as 97.5% w/w, and most preferably 100% w/w, based on the total amount of disintegrant being present in the tablet.
16. The process according to any of the preceding items, wherein the total amount of disintegrants(s) in the tablet is 0.5-10% w/w, 1-9% w/w, or 2-8% w/w, such as 3-6% w/w, based on the weight of the tablet.
17. The process according to any of the preceding items, wherein the total amount of filler(s), such as microcrystalline cellulose, in the tablet is 60-85% w/w, 70-85% w/w, or 70-80% w/w, based on the weight of the tablet.
18. The process according to any of the preceding items, wherein the solid form of an API is a freely soluble solid form of an API.
19. The process according to any of the preceding items, wherein the compression in step c) is carried out by applying a compression force of 6 N per mm² to 100 N per mm², preferably of from 8 N per mm² to 50 N per mm², such as from 10 N per mm² to 25 N per mm².
20. The process according to any of the preceding items, wherein the solid form of said very soluble or freely soluble API is a solid form of elagolix or eravacycline.
21. Process for the preparation of a coated tablet comprising the preparation of a tablet according to any one of items 1 to 20, further comprising a step of coating the tablets.
22. The process according to item 21, wherein the coating is a gastroresistant coat or a non-gastroresistant coat.
23. The process according to item 22, wherein the coating is a gastroresistant coat and the the solid form of the API is very soluble or freely soluble in FASSIF-medium according to European Pharmacopoeia 9.4, Section 5.11, Character Section in Monograph.
24. The process according to item 21, wherein the coating is a non-gastroresistant coat and the solid form of the API is very soluble or freely soluble in 0.1N HCl-solution according to European Pharmacopoeia 9.4, Section 5.11, Character Section in Monograph.
25. Compressed rapidly or very rapidly dissolving tablet comprising a very soluble or freely soluble solid form of an active pharmaceutically ingredient (API), wherein the tablet comprises a mixture of a granulate and a tablet base, wherein
the granulate comprises a very soluble or freely soluble solid form of an active pharmaceutically ingredient (API) in form of granules,
   wherein said granulate comprises at least 90% w/w of the total amount of said very soluble or freely soluble solid form of the API being present in the tablet; and
the tablet base comprises one or more disintegrants and optionally further excipients, wherein the amount of said very soluble or freely soluble solid form of said API in the tablet is at least 10% w/w based on the weight of the tablet and wherein, when the tablet is broken in half by hand and the surface of the break is analyzed, at least five of said granules have an apparent surface area of at least 30000 μm².
   26. Compressed rapidly or very rapidly dissolving tablet comprising a very soluble or freely soluble solid form of an active pharmaceutically ingredient (API), wherein the tablet comprises a mixture of a granulate and a tablet base, wherein
the granulate comprises a very soluble or freely soluble solid form of an active pharmaceutically ingredient (API) in form of granules, wherein said granulate comprises at least 90% w/w of the total amount of said very soluble or freely soluble solid form of the API being present in the tablet; and
the tablet base comprises one or more disintegrants and optionally further excipients and/or APIs,
   wherein the amount of said very soluble or freely soluble solid form of said API in the tablet is at least 10% w/w based on the weight of the tablet and wherein, when the tablet is broken in half by hand and the surface of the break is analyzed, at least five of said granules have an apparent circumference of at least 600 μm.
   27. Compressed rapidly or very rapidly dissolving tablet comprising a very soluble or freely soluble solid form of an active pharmaceutically ingredient (API), wherein the tablet comprises a mixture of a granulate and a tablet base, wherein
the granulate comprises a very soluble or freely soluble solid form of an active pharmaceutically ingredient (API) in form of granules, wherein said granules comprise at least 90% w/w of the total amount of said very soluble or freely soluble solid form of the API being present in the tablet; and
the tablet base comprises one or more disintegrants and optionally further excipients and/or APIs,
   wherein the amount of said very soluble or freely soluble solid form of said API in the tablet is at least 10% w/w based on the weight of the tablet, wherein at least 90% of said granules have a size of at least 160 μm, preferably at least 240 μm, such as at least 320 μm, at least 400 μm, at least 450 μm,
   preferably wherein the size is at most 1000 μm.
   28. The tablet according to items 25 to 27, wherein the amount of said very soluble or freely soluble solid form of an API in said granules is at least 50% w/w, based on the total weight of said granules; preferably the amount of said very soluble or freely soluble solid form of an API in said granules is at least 60% w/w or at least 70% w/w; more preferably at least 80% w/w, at least 90% w/w or at least 95% w/w; respectively based on the total weight of said granules; and most preferably, said granules essentially consist of said very soluble or freely soluble solid form of an API.
   29. The tablet according to items 25 to 28, wherein
   a) the solid form of the API is very soluble or freely soluble in FASSIF-medium according to European Pharmacopoeia 9.4, Section 5.11, Character Section in Monograph; or
   b) the solid form of the API is very soluble or freely soluble in 0.1N HCl-solution according to European Pharmacopoeia 9.4, Section 5.11, Character Section in Monograph.
   30. The tablet according to any of items 25-29, wherein the amount of said very soluble or freely soluble solid form of said API in the tablet is from 15% w/w to 60% w/w, such as from 15% w/w to 50% w/w, such as from 15% w/w to 40% w/w, for example from 20% w/w to 60% w/w, from 20% w/w to 50% w/w, such as from 20% w/w to 40% w/w, based on the total weight of the tablet.
   31. The tablet according to any of items 25-30, wherein the tablet comprises at least one further solid form of an API, wherein said further solid form of an API is intragranular when the further solid form of an API is a very soluble or freely soluble solid form.
   32. The tablet according to any of items 25-31, wherein the granulate has been prepared by a dry or wet granulation process.
   33. The tablet according to any of items 25-32, wherein the granulate comprises at most 10% w/w disintegrant(s), such as at most 7% w/w, or at most 5% w/w, or at most 2.5% w/w, based on the total amount of disintegrants being present in the tablet, preferably wherein the granulate comprises no disintegrants.
   34. The tablet according to any of items 25 to 33, wherein the granules of step (a) comprise at most 10% w/w, such as at most 7% w/w, or at most 5% w/w, or at most 2.5% w/w disintegrant(s), based on the total weight of the granules.
   35. The tablet according to any of items 25-34, wherein the amount of said very soluble or freely soluble solid form of said API in the granulate is at least 90% w/w, preferably at least 93% w/w, more preferably at least 95% w/w, such as 97.5% w/w, and most preferably 100% w/w, based on the total amount of API being present in the tablet.
   36. The tablet according to any of items 25-35, wherein the tablet base comprises at least 90% w/w, preferably at least 93% w/w, more preferably at least 95% w/w, such as 97.5% w/w, and most preferably 100% w/w, based on the total amount of disintegrant being present in the tablet.
   37. The tablet according to any of items 25-36, wherein the API is a freely soluble API.
   38. The tablet according to any of items 25-37, wherein the tablet is characterized by a hardness of 25N-500N, more preferably 50N-300N.
   39. The tablet according to any of items 25-38, wherein the optionally further pharmaceutically acceptable excipients are selected from the group consisting of fillers, glidants, and lubricants.
   40. The tablet according to item 39, wherein the filler is at most sparingly soluble as determined according to European Pharmacopoeia 9.4, Section 5.11, Character Section in Monograph; more preferably wherein the filler is microcrystalline cellulose.
   41. The tablet according to item 39 or 40, wherein the glidant is selected from the group consisting of starch, talcum, colloidal silica i.e. syloid, pyrogenic silica, hydrated sodium silioaluminate, and highly dispersed silicone dioxide (e.g., sold under the trade name “Aerosil®)”; more preferably, the glidant is highly dispersed silicone dioxide (e.g., sold under the trade name “Aerosil®”.
   42. The tablet according to any one of items 39 to 41, wherein the lubricant is selected from the group consisting of magnesium stearate, calcium stearate, sodium stearate, boric acid, sodium benzoate, sodium oleate, sodium lauryl sulfate (SLS), magnesium lauryl sulfate (MLS), and sodium fumarate; more preferably wherein the lubricant is magnesium stearate.
   43. The tablet according to any of items 25 to 42, wherein the disintegrant is one or more selected from the group consisting of starch, crosslinked cellulose, crosslinked polyvinylpyrrolidone (PVP), crosslinked alginic acid, and ion exchange resin; preferably wherein the disintegrant is croscarmellose sodium.
   44. Coated tablet comprising the rapidly or very rapidly dissolving tablet according to any of items 25-43, and/or prepared by a process according to any one of items 1 to 24.
   45. The tablet according to item 44, wherein the coat is a gastroresistant coat or a non-gastroresistant coat.
   46. The process according to any of items 1 to 24, the tablet according to any of items 25 to 43, and the coated tablet according to item 44 or 45, wherein the API is a tetracycline, in particular wherein the tetracycline is eravacycline or omadacycline.
   47. The process according to any of items 1 to 24, the tablet according to any of items 25 to 43, and the coated tablet according to item 44 or 45, wherein the API is amorphous or crystalline eravacycline dihydrochloride.
   48. The process according to any of items 1 to 24, the tablet according to any of items 25 to 43, and the coated tablet according to item 44 or 45, wherein the API is a GnRH antagonist, in particular wherein the GnRH antagonist is elagolix.
   49. The process according to any of items 1 to 24, the tablet according to any of items 25 to 43, and the coated tablet according to item 44 or 45, wherein the API is amorphous or crystalline elagolix sodium salt.
   50. The process, tablet or the coated tablet according to item 49, wherein the API is amorphous elagolix sodium salt, the total amount of disintegrants in the tablet is 3-6% w/w, based on the weight of the tablet, and the wherein the total amount of filler(s) in the tablet is 70-80% w/w, based on the weight of the tablet.
   51. The process, tablet or the coated tablet according to item 47, wherein the API is crystalline eravacycline dihydrochloride, the total amount of disintegrants in the tablet is 5-10% w/w, based on the weight of the tablet, and the wherein the total amount of filler(s) in the tablet is 65-75% w/w, based on the weight of the tablet.

Other objects, features, advantages and aspects of the present invention will become apparent to those of skill from the following description. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and the other parts of the present disclosure.

### Definitions

In the context of the present invention the following abbreviations have the indicated meaning, unless explicitly stated otherwise:

As used herein, a tablet comprising a ‘very soluble’ or ‘freely soluble’ solid form of an active pharmaceutically ingredient (API) is defined as ‘rapidly dissolving’ when a mean (of e.g., six tested tablets) of at least 85 percent of said active pharmaceutical ingredient dissolves within 30 minutes, using United States Pharmacopeia (USP) Apparatus 1 at 100 rpm at 37° C. in a volume of 500 ml of at least one of
FASSIF (‘Fasted-State Stimulated Intestinal Fluid’)-medium; and
0.1N HCl-solution.

A tablet is ‘very rapidly’ dissolving when a mean of at least 85 percent of said active pharmaceutical ingredient dissolves within 15 minutes in the above test.
FASSIF-medium:
Sodium taurocholate 3 mM
Lecithin 0.75 mM
NaOH (pellets) 8.7 mM [0.174 g]
NaH₂PO₄.H₂O 28.4 mM [1.977 g]
NaCl 106 mM [3.093 g]
Purified water qs. 500 mL

FASSIF-medium has a pH of 6.50 and an osmolality of about 270 mOsmol/kg.

As used herein, a solid form of an API is “very soluble” if the API is completely dissolved when carrying out the following procedure according to the European Pharmacopoeia 9.4, Section 5.11, Character Section in Monograph:
Weigh 100 mg of finely powdered substance (API) in a stoppered tube (16 mm in internal diameter and 160 mm long), add 0.1 mL of the solvent (which is either the FASSIF medium or the 0.1 HCl-solution) and proceed as follows (“Dissolving Procedure”):
   Shake vigorously for 1 min and place in a constant temperature device, maintained at a temperature of 25.0+/−0.5° C. for 15 min. If the substance is not completely dissolved, repeat the shaking for 1 min. and place the tube in the constant temperature device for 15 min.
If the substance (API) is completely dissolved, it is “very soluble”.
If the substance (API) is not completely dissolved, add 0.9 mL of the same solvent and proceed with the “Dissolving Procedure” described above
If the substance is then completely dissolved, it is “freely soluble”.

The term “gastroresistant coating” as used herein is interchangeable with the term “enteric coating” and it refers to a coating that does not allow acidic gastric juice to penetrate through but it allows the penetration of water to the disintegrant-containing tablet (e.g., by dissolution, swelling, degradation etc.) at the essentially neutral pH of the intestines. In particular, a pH-dependent gastroresistant polymer suitable for purposes of the present invention is a polymer which dissolves, swells or degrades at a pH of 4.5 or higher, preferably pH 5.0 or higher. In a typical embodiment, the polymer dissolves, swells or degrades at a pH in the range of from 4.5 to 7.0, preferably from 5.0 to 6.5. Non-limiting examples of suitable pH-dependent entero-resistant polymers useful as the coating material for purpose of the present invention include, alone or in combination, cellulose esters such as cellulose acetate phthalate, hydroxypropyl methylcellulose acetate succinate or cellulose acetate succinate; and/or methacrylic acid copolymers such as those sold as Eudragit® L, Eudragit® S by Evonik or Acryleze® by Colorcon. Preferably, the polymer is hydroxypropyl methylcellulose acetate succinate and/or methacrylic acid-ethyl acrylate copolymer. Most preferably, the polymer in the gastroresistant coating is methacrylic acid-ethyl acrylate copolymer (1:1). Gastroresistant coating can comprise at least one polymer being insoluble in aqueous solutions having pH value of less than 4.5, such as the polymers mentioned above, and at least one further excipient selected from plasticizers, antitacking agents, pigments, colorants and/or surface active substances. The thickness of the gastroresistant coating is preferably in the range of 40 to 250 μm, preferably 45 to 200 μm and most preferably 50 to 180 μm.

The term “intragranular” as used herein defines a part of a pharmaceutical composition (such as a tablet) that is within a granule.

The term “tablet base” as used herein refers to the part of the tablet that is not a granule. In particular, the tablet base comprises one or more disintegrant(s) and one or more filler(s).

The term “granulate” as used herein defines a plurality of granules. The term “granule” (plural “granules”) as used herein has the common meaning and particularly refers to a multiparticle entity of agglomerated small particles, wherein said multiparticle entity is substantially spherical and can be obtained by dry or wet granulation techniques known in the art. The granules form the part of the tablet that is not the tablet base. The granules comprise at least 90% w/w of the total amount of the very soluble or freely soluble solid form of the API that is present in the tablet. Preferably, the granules are homogenously distributed in the tablet base due to a step of mixing the granules with the tablet base before compression of the mixture into the tablet.

The expression “granulate comprising said very soluble or freely soluble API in form of granules, wherein at least 90% of said granules have a size of at least 150 μm” as used herein refers to the percentage of granules of said granulate that is retained by a sieve having an ISO Nominal Aperture of 150 μm. The granulate to be used in the process of the present invention can be tested for having the required size distribution as defined herein (e.g., at least 90% of said granules have a size of at least AAAμm) by the European Pharmacopoeia 9.0 method 2.9.38. “Particle size distribution estimation by analytical sieving”. Briefly, sieving is carried out under conditions that do not cause the test sample to gain or lose moisture. The relative humidity of the environment in which the sieving is carried out must be controlled to prevent moisture uptake or loss by the sample. In the absence of evidence to the contrary, analytical test sieving is normally carried out at ambient humidity, e.g. 40% r.h. (r.h.=rh=relative humidity), and at ambient temperature, e.g. at 20° C. A test sample having a mass of 25 g to 100 g is typically used for test sieves having a 200 mm diameter. A dry sieving method with mechanical agitation is employed. Preferred is the apparatus Retsch AS 300 control B with an amplitude setting of 0.4 mm, as employed in the examples of the present invention.

“AAAμm” as used herein defines a desired target size of the granules that are comprised in the granulate of the present invention. For example, it can be desired in the present invention that the granulate comprises granules, wherein at least 90% of said granules have a size of at least 150 μm. In this case, the desired target size of at least 90% of the granules is 150 μm. It can also be desired that at least 90% of the granules have a size of at least 200 μm. Then, the desired target size is 200 μm.

The term “essentially” in the context of the present invention denotes (unless indicated differently) an interval of accuracy that the person skilled in the art will understand to still ensure the technical effect of the feature in question. The term typically indicates deviation from the indicated numerical value of ±1%, and preferably ±0.5%.

Sieves are selected to cover the entire range of particle sizes present in the test sample, wherein the sieve having a mesh size of 150 μm is the finest sieve at the bottom of a nest of sieves having a recommended square-root 2 progression of the area of the sieve openings. All sieves are weighed beforehand. The sample weight is also determined prior to sieving. The sample is placed on the uppermost sieve of the nest of sieves and sieving is carried out for 5 minutes, when all sieves are weighed. Sieving is continued for 5 more minutes and the sieves are weighed again. Sieving is continued until the mass on all of the test sieves no longer changes by more than 5%. When constant values are achieved all sieves and the collection container below the smallest sieve collecting the fine particles are weighed.

If 90% w/w or more of the tested sample are retained in the sieves having a mesh size of 150 μm or more, then within the meaning of the present invention, a sample consists of “granules, wherein at least 90% w/w of said granules have a size of at least 150 μm”. Preferably more than 90% w/w of the tested sample are retained in the sieves having a mesh size of 150 μm or more, and more preferably more than 95% w/w of the tested sample are retained in the sieves having a mesh size of 150 μm or more. Most preferred, all granules of the tested sample are retained in the sieves having a mesh size of 150 μm or more.

### BRIEF DESCRIPTION OF THE DRAWINGS

FIG. 1: This figure shows the dissolution profiles of tablets containing compressed pure granulated (250 bar granulation pressure) and fractionated elagolix sodium (the API), with the fractions of granulated API being >500 μm; 500 μm-250 μm; 250 μm-150 μm; <150 μm. The x-axis denotes time in minutes, the y-axis the percentage of dissolved API at a given point in time. The release (dissolution)-profiles were obtained as described in the examples. Tablets prepared from the largest API granules showed the fastest dissolution.

FIG. 2: This figure is a comparison of the dissolution-profile of tablets containing compressed pure granulated elagolix sodium (from a size fraction 800 μm>granules>250 μm) obtained when applying different granulation pressures of 150 bar, 200 bar, and 250 bar. Additionally depicted is the dissolution-profile of tablets prepared from ungranulated elagolix sodium. The x-axis denotes time in minutes, the y-axis the percentage of dissolved API at a given point in time. The release (dissolution)-profiles were obtained as described in the examples. The tablets prepared from large elagolix granules demonstrated fast dissolution independently from the tested granulation pressure, and dissolution was significantly improved compared to tablets prepared from non-granulated elagolix.

FIG. 3: This figure shows the dissolution profiles of tablets containing compressed pure granulated and fractionated amorphous eravacycline dihydrochloride (the API), with the fractions of granulated API being >500 μm; 500 μm-250 μm; 250 μm-150 μm; <150 μm. The x-axis denotes time in minutes, the y-axis the percentage of dissolved API at a given point in time. The release (dissolution)-profiles were obtained as described in the examples. Tablets prepared from the largest eravacycline granules showed the fastest dissolution.

FIG. 4: This figure shows the dissolution profiles of tablets containing compressed pure granulated and fractionated crystalline eravacycline dihydrochloride (the API), with the fractions of granulated API being >500 μm; 500 μm-250 μm; 250 μm-150 μm; <150 μm. The x-axis denotes time in minutes, the y-axis the percentage of dissolved API at a given point in time. The release (dissolution)-profiles were obtained as described in the examples. Tablets prepared from the eravacycline granules having a size in the range from 250 μm to 500 μm showed the fastest dissolution.

FIG. 5: Detection of coloured granules in a black and white photo of an elagolix tablet prepared from large granules that was broken apart. The spatial separation of the light grey API-containing granules from the tablet base comprising the disintegrant is apparent. The white bar to the bottom right indicates a length of 100 μm for scale.

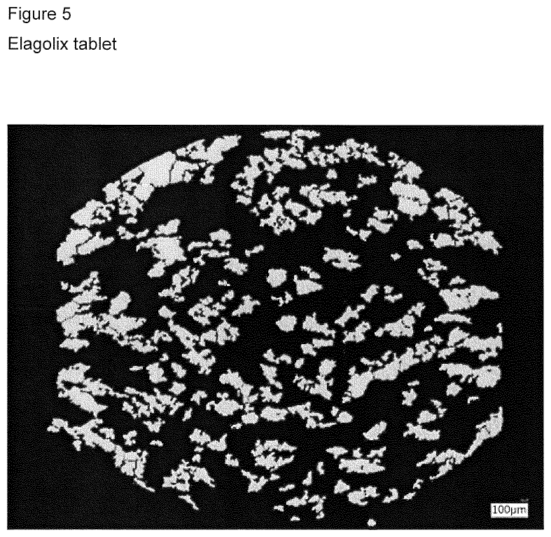

FIG. 6: Granules of crystalline eravacycline dihydrochloride (yellow granules appear grey in the b/w photo) are visible to the naked eye in tablets prepared according to the process of the present invention. Granule size increases from left to right.

FIG. 7: Recovery of Elagolix after stress test of tablets comprising granules of elagolix sodium. The x-axis denotes duration of stress test, the y-axis the percentage of recovered API at a given time point.

FIG. 8: This figure shows the dissolution profiles of tablets containing compressed pure granulated and fractionated elagolix sodium (the API) after stress tests at 25° C./60% rh. The x-axis denotes time in minutes, the y-axis the percentage of dissolved API at a given point in time. The release (dissolution)-profiles were obtained as described in the examples. No change in the dissolution kinetic was observed after the stress test.

### DETAILED DESCRIPTION OF THE INVENTION

The invention is described below in further detail by embodiments, without being limited thereto.

In one aspect, the present invention relates to a process for the preparation of a rapidly or very rapidly dissolving tablet comprising a very soluble or freely soluble solid form of an active pharmaceutically ingredient (API), with this API being present in the tablet in a minimum amount of at least 10% w/w based on the weight of the tablet, wherein the tablet comprises a granulate and a tablet base, wherein said granulate comprises granules comprising at least 90% w/w of the total amount of said very soluble or freely soluble form of the API being present in the tablet, and wherein the tablet base comprises one or more disintegrants.

The present inventors have found that tablets comprising at least freely soluble API can have problems with tablet disintegration and thus API dissolution in in vitro dissolution assays. This problem becomes more pronounced as the drug load of such tablets increases, for example to drug loads of at least 10% w/w API based on the total weight of the tablet. Tablets formulated according to a standard IR formulation and containing at least 10% elagolix, for example, did not qualify as rapidly dissolving or very rapidly dissolving, even though they contained sufficient amounts of disintegrant. The present inventors have successfully addressed this problem.

It has been surprisingly found in the context of the present invention that the spatial separation of a very soluble or freely soluble solid form of an API from (the) disintegrant(s) leads to faster tablet disintegration and thus dissolution of the at least freely soluble solid form of the API. Without wishing to be bound to any theory, it is believed that this spatial separation decreases the competition between disintegrant and said API for water in an aqueous environment, which could delay disintegration of the tablet if the disintegrant cannot swell sufficiently. By having said very soluble or freely soluble solid form of an API and the disintegrant in separate areas of the pharmaceutical composition (or dosage form), that is, in the API-containing granulate and in the disintegrant-containing tablet base respectively, it is believed that the disintegrants in the tablet base come into a more intense contact with water first, prior to said API in the granulate. This results in the fast disintegration of the dosage form and—since the solid form of the API is at least freely soluble—finally in a comparably rapid dissolution of said API.

Thus, by applying the process of the present invention, tablets are provided that are rapidly or very rapidly dissolving, despite the comparatively high drug load of at least 10% w/w API based on the total weight of the tablets.

The present invention thus relates to a process for the preparation of a rapidly or very rapidly dissolving tablet comprising a very soluble or freely soluble solid form of an active pharmaceutically ingredient (API) as defined in the items which comprises at least steps (a)-(c), in this order.

In step a) of the process of the present invention, a granulate is prepared comprising said very soluble or freely soluble solid form of an API in form of granules, wherein at least 90% w/w of said granules have a size of at least 150 μm. The granulation method, for example dry granulation, can be adapted so as to already produce a granulate comprising granules exhibiting the desired size distribution.

This can be confirmed analytically by the European Pharmacopoeia 9.0 sieving method 2.9.38. described in more detail below.

The particle size distribution of the granules can be further controlled by sieving the granules with the respective sieves having a suitable mesh size, such as an open mesh size of 150 μm, 200 μm, 300 μm, 400 μm, 500 μm or 800 μm, so as to remove most of the fines or small granules having a size that is not retained on the respective sieve. Thus, by carrying out the European Pharmacopoeia 9.0 sieving method 2.9.38, it can be controlled whether the feature “granules, wherein at least 90% w/w of said granules have a size of at least 150 μm” is fulfilled. Likewise, any further particle size distribution disclosed herein can be controlled.

An API, like elagolix, can come in different forms, such as the free form of the API, e.g. uncharged elagolix, or salts of the API, such as elagolix sodium. These forms can in turn form different solid forms, such as amorphous or crystalline solid forms, and the crystalline forms can demonstrate polymorphism, i.e. they sometimes form crystals that can be distinguished by physicochemical properties.

The different solid forms of an active pharmaceutical ingredient can thus have different physicochemical properties, such as different solubilities. For example the amorphous form of an API is often more soluble than the crystalline form. In the context of the present invention “a very soluble or freely soluble solid form of an active pharmaceutically ingredient (API)” refers to the solubility of that particular solid form of an API which is actually to be used in the preparation of the granulate of step a).

In step b), the granulate obtained in step a) is mixed with the tablet base comprising one or more disintegrants and optionally further excipients and/or APIs. Thus, the mixture obtained from step b) is inhomogeneous in that most of the API is in the granulate while most of the disintegrant is extragranular.

In step c), the mixture from step b) is compressed to form said tablet.

It is possible to apply a compression force of 6 N per mm² to 100 N per mm², preferably of from 8 N per mm² to 50 N per mm², such as from 10 N per mm² to 25 N per mm².

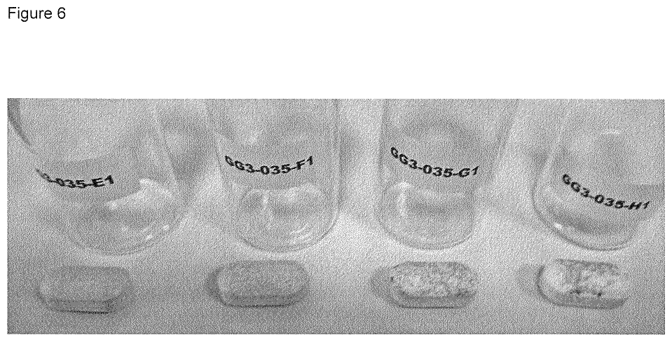

Step c) forms tablets where the granulate is embedded in a tablet base which is distinct from the granulate. For example FIG. 6 shows tablets comprising granulate comprising eravacycline granules which are readily detectable as visibly distinct from the tablet base because in this particular example the granules comprising the yellow API can be readily distinguished from the white tablet base.

In the process and the tablet of the present invention, the granulate comprises most of the API being present in the tablet to achieve a predominant separation of API and disintegrant. Typically the granulate comprises at least 90% w/w of the total amount of said very soluble or freely soluble solid form of the API being present in the tablet. The amount of said very soluble or freely soluble solid form of said API in the tablet is at least 10% w/w based on the weight of the tablet. The tablet base comprises one or more disintegrants.

The API which is used in the process, tablet and coated tablet of the invention is ‘very soluble’ or ‘freely soluble’ under conditions in the intestines or stomach, depending on where the tablet shall dissolve, wherein ‘very soluble’ or ‘freely soluble’ is defined as follows:

a) the solid form of the API (in neat form) is very soluble or freely soluble in FASSIF-medium according to European Pharmacopoeia 9.4, Section 5.11, Character Section in Monograph (for the purpose of the present invention taken as representative of intestine conditions; normally the pH value for the intestine is in the range of 5.0 to 8.0, depending e.g. on the state of saturation); or
b) the solid form of the API (in neat form) is very soluble or freely soluble in 0.1N HCl-solution according to European Pharmacopoeia 9.4, Section 5.11, Character Section in Monograph (for the purpose of the present invention taken as representative of stomach conditions; normally the pH value for the stomach is in the range of 0.8 to 1.5). If the tablet shall dissolve in the intestines, an enteric coating can be applied onto the tablet to avoid premature dissolution in the stomach. If an immediate release coating or no coating is provided onto the tablet, the tablet may dissolve in the stomach. An immediate release coating is preferred if dissolution in the stomach is desired so as to avoid premature dissolution in the mouth.

Accordingly, if a coating is used, the coating can be a gastroresistant coat or a non-gastroresistant coat.

In one aspect, the solid form of an API is a freely soluble solid form of an API. The solid form of said very soluble or freely soluble API may be a solid form of elagolix or eravacycline, such as elagolix sodium or eravacycline dihydrochloride.

The coating may be a gastroresistant coat and the solid form of the API a very soluble or freely soluble in FASSIF-medium according to European Pharmacopoeia 9.4, Section 5.11, Character Section in Monograph. This combination can for example be chosen if it is desired to prepare a rapidly or very rapidly dissolving tablet wherein the API is to be released in the intestine.

The coating can also be a non-gastroresistant coat or no coat and the solid form of the API is very soluble or freely soluble in 0.1N HCl-solution according to European Pharmacopoeia 9.4, Section 5.11, Character Section in Monograph. This combination can for example be chosen if it is desired to prepare a rapidly or very rapidly dissolving tablet wherein the API is to be released in the stomach.

The amount of the very soluble or freely soluble solid form of said API in the tablet can be at least 10% w/w, such as at least 15% w/w, such as at least 20% w/w, at least 25% w/w, at least 30% w/w, or at least 35% w/w, based on the total weight of the tablet.

The amount of said very soluble or freely soluble solid form of said API in the tablet can also be from 10% w/w to 60% w/w, such as from 10% w/w to 50% w/w, such as from 15% w/w to 40% w/w, for example from 20% w/w to 60% w/w, from 20% w/w to 50% w/w, such as from 20% w/w to 40% w/w, based on the total weight of the tablet.

The tablet may further comprise at least one further solid form of an API, such as a solid form of a different API, wherein said further solid form of an API is intragranular when the further solid form of an API is a very soluble or freely soluble solid form. The skilled person will appreciate that the concept of keeping very soluble or freely soluble solid forms of an API apart from the disintegrant in the tablet base also applies to the situation where two or more soluble APIs are present in the tablet.

The granules of step (a) can have a size of at least 150 μm, at least 200 μm, preferably at least 300 μm, more preferably at least 400 μm, and further preferred at least 500 μm, wherein the size is preferably at most 1500 μm or at most 1200 μm, for example the size is in a range from 200 μm to 1500 μm or to 1200 μm, or from 300 μm to 1500 μm or to 1200 μm, or from 400 μm to 1000 μm, preferably in a range from 500 μm to 1500 μm. In other words, e.g. at least 90% w/w of said granules have a size of at least 200 μm, preferably at least 300 μm, more preferably at least 400 μm, and further preferred at least 500 μm, wherein preferably e.g. at least 90% w/w of said granules have a size of at most 1500 μm or at most 1200 μm. For example, at least 90% w/w of said granules have a size in a range from 200 μm to 1500 μm or to 1200 μm, or from 300 μm to 1500 μm or to 1200 μm, or from 400 μm to 1000 μm, preferably in a range from 500 μm to 1500 μm.

The tablet obtained after compression may comprise granulate comprising said very soluble or freely soluble solid form of an active pharmaceutically ingredient (API) in form of granules wherein, when the tablet is broken in half by hand and the surface of the break is analyzed, at least five of said granules, such as at least 20 of said granules, or for example at least 50 of said granules, have an apparent surface area of at least 30000 μm².

In step (a) the granules can be prepared by carrying out a dry or wet granulation process. The granules are preferably prepared by carrying out a dry granulation process, for example by using a slugging technique or by using a compactor such as a roller compactor, preferably by using roller compaction. The roller compaction can be performed as commonly known in the art and is essentially performed as follows: conveying powdered API to the compaction area, normally with a screw feeder, compacting powder between two counter-rotating rolls with applied forces (roller compactor, e.g., chilsonator) into a ribbon or small pellets, and milling the resulting compact to obtain a desired particle size distribution. Alternatively, a large tablet (slug) is produced in a heavy duty tabletting press and these tablets are milled through a low-shear mill.

The granulate of step (a) may comprise at most 10%, such as at most 7%, or at most 5%, or at most 2.5% w/w disintegrants, based on the total amount of disintegrant being present in the tablet, and preferably the granulate of step (a) comprises no disintegrant. In other words, it is desired, but not essential, to have no disintegrant in the API-containing granulate.

In a preferred embodiment, the granulate of step (a) comprise at most 10% w/w, such as at most 7% w/w, or at most 5% w/w, or at most 2.5% w/w disintegrant(s), based on the total weight of the granulate.

The amount of said very soluble or freely soluble solid form of said API in the granulate of step a) can be at least 90% w/w, such as at least 93% w/w, preferably at least 95% w/w, more preferably at least 97.5% w/w, and most preferably 100% w/w, based on the total amount of API being present in the tablet. The preparation and use of granulate comprising granules consisting essentially of API only allows for a particularly easy preparation process wherein only API is used in step a).

In a preferred embodiment, the amount of said very soluble or freely soluble solid form of said API in the granulate of step (a) is at least 50% w/w; preferably at least 60% w/w or at least 70% w/w; more preferably at least 80% w/w, at least 90% w/w or at least 95% w/w; based on the total weight of the granulate; and most preferably, said granulate essentially consist of said very soluble or freely soluble solid form of an API. Providing granulate exhibiting said minimum amount of said very soluble or freely soluble solid form of said API in the granulate of step (a) additionally enhances the desired spatial separation of said API and disintegrant, thereby further contributing to the advantageous properties of the tablet.

The tablet base comprises a disintegrant in an amount sufficient to rapidly or very rapidly disintegrate the granulate-containing tablet. The total amount of disintegrant(s) in the tablet can be, for example, 0.5-15% w/w, 1-10% w/w, or 3-7% w/w, such as 5% w/w, based on the weight of the tablet.

The tablet base may comprise at least 90% w/w, preferably at least 93% w/w, more preferably at least 95% w/w, such as 97.5% w/w, and most preferably 100% w/w of the desintigrant, based on the total amount of disintegrant being present in the tablet.

The total amount of filler(s), such as microcrystalline cellulose, in the tablet may be, for example, 25-89.5% w/w, such as 40-89% w/w, for example 43-77% w/w or 53-77% w/w, based on the weight of the tablet. The skilled person will appreciate that the relative amount of filler in the formulation can be adjusted to the relative amount of API in the formulation in what is an inverse relationship.

The tablet base typically comprises at least 80% w/w filler(s), preferably at least 90% w/w, or at least 95% w/w, or 100% w/w, based on the total amount of filler(s) being present in the tablet. In other words, it is desired, but not essential, to have no filler in the API-containing granulate.

The majority of filler(s) and disintegrant(s) should be present in the tablet base. Ideally no filler and no disinregrant should be present in the API-containing granules. However, it is not essential that no filler and no disintegrant are present in the granules, smaller amounts of filler(s) and/or disintegrant(s) may be present in the granules.

In a further embodiment, the compression in step c) of the process according to the present invention is carried out by applying a compression force of 6 N per mm² to 100 N per mm², preferably of from 8 N per mm² to 50 N per mm², such as from 10 N per mm² to 25 N per mm².

The solid form of the API can be an amorphous or crystalline form. Preferably, the solid form of elagolix is an amorphous form, preferably it is amorphous elagolix sodium salt. Preferably, the solid form of eravacycline is a crystalline form, preferably it is crystalline eravacycline dihydrochloride.

Elagolix sodium can be obtained for instance as disclosed in WO 2009062087, example 5. Eravacycline dihydrochloride can be obtained for instance as disclosed in WO 2014/036502, scheme 9.

The invention also refers to the preparation of a coated tablet, the process comprising the process of preparing tablets according to the present invention and further comprising a step of coating the tablets. The type of coating depends e.g. on the intended release site of the API. If it is desired that the API is released in the stomach, then a non-gastroresistant coating can be applied. Such a non-gastroresistant coating allows for the release of the API in the stomach.

If it is desired to release the API after the stomach passage, a gastroresistant coating can be applied. Such a gastroresistant coating prevents the release of the API in the stomach and thus e.g. allows for the release of the API after the stomach passage.

The invention further relates to a compressed rapidly or very rapidly dissolving tablet comprising a very soluble or freely soluble solid form of an active pharmaceutically ingredient (API), wherein the tablet comprises a mixture of granulate and a tablet base, wherein
the granulate comprises a very soluble or freely soluble solid form of an active pharmaceutically ingredient (API) in form of granules, wherein said granulate comprises at least 90% w/w of the total amount of said very soluble or freely soluble form of the API being present in the tablet; and
the tablet base comprises one or more disintegrants and optionally further excipients, wherein the amount of said very soluble or freely soluble solid form of said API in the tablet is at least 10% w/w based on the weight of the tablet and wherein, when the tablet is broken in half by hand and the surface of the break is analyzed, at least five of said granules have an apparent surface area of at least 30000 μm², or at least 40000 μm², such as at least 50000 μm². Preferably at least 20 of said granules, for example at least 50 of said granules, have an apparent surface area of at least 30000 μm², or at least 40000 μm², such as at least 50000 μm².

The invention also relates to a compressed rapidly or very rapidly dissolving tablet comprising a very soluble or freely soluble solid form of an active pharmaceutically ingredient (API), wherein the tablet comprises a mixture of a granulate and a tablet base, wherein
the granulate comprises a very soluble or freely soluble solid form of an active pharmaceutically ingredient (API) in form of granules, wherein said granulate comprise at least 90% w/w of the total amount of said very soluble or freely soluble form of the API being present in the tablet; and
the tablet base comprises one or more disintegrants and optionally further excipients and/or APIs,
   wherein the amount of said very soluble or freely soluble solid form of said API in the tablet is at least 10% w/w based on the weight of the tablet and wherein, when the tablet is broken in half by hand and the surface of the break is analyzed, at least five of said granules have an apparent circumference of at least 600 μm, such as at least 800 μm, or at least 1000 μm. Preferably at least 20 of said granules, for example at least 50 of said granules, have an apparent circumference of at least 600 μm, such as at least 800 μm, or at least 1000 μm.

As described above, the terms ‘very soluble’ and ‘freely soluble’ as defined as follows:

a) the solid form of the API is very soluble or freely soluble in FASSIF-medium according to European Pharmacopoeia 9.4, Section 5.11, Character Section in Monograph; or
b) the solid form of the API is very soluble or freely soluble in 0.1N HCl-solution according to European Pharmacopoeia 9.4, Section 5.11, Character Section in Monograph. In one embodiment, the API is a freely soluble API.

An alternative to measuring the surface area or circumference of the granules is measuring the longest diameter of the granules, which, after compression to form the tablets, is a bit smaller than the longest diameter of the granules which were used for step b) of the process for the preparation of the tablets. The granules can have a longest diameter of at least 160 μm, preferably at least 240 μm, equally preferred at least 320 μm, further preferred at least 400 μm, more preferred at least 450 μm, wherein the size is preferably at most 1000 μm, equally preferred at most 900 μm or 850 μm, for example the size is in a range from 160 μm to 1000 μm, or from 240 μm or 320 μm to 900 μm, or from 400 μm to 850 μm, preferably in a range from 450 μm to 800 μm.

The amount of said very soluble or freely soluble solid form of said API in the tablet may be at least 10% w/w, for example at least 15% w/w, such as at least 20% w/w, at least 25% w/w, at least 30% w/w, or at least 35% w/w, based on the weight of the tablet.

The amount of said very soluble or freely soluble solid form of said API in the tablet can also be from 15% w/w to 60% w/w, such as from 15% w/w to 50% w/w, such as from 15% w/w to 40% w/w, for example from 20% w/w to 60% w/w, from 20% w/w to 50% w/w, such as from 20% w/w to 40% w/w, based on the total weight of the tablet.

In a preferred embodiment, the amount of said very soluble or freely soluble solid form of an API in said granules is at least 50% w/w, based on the total weight of said granules; preferably the amount of said very soluble or freely soluble solid form of an API in said granules is at least 60% w/w or at least 70% w/w; more preferably at least 80% w/w, at least 90% w/w or at least 95% w/w; respectively based on the total weight of said granules; and most preferably, said granules essentially consist of said very soluble or freely soluble solid form of an API. Granules exhibiting said minimum amount of said very soluble or freely soluble solid form of said API in the granules of step (a) additionally enhance the desired spatial separation of said API and disintegrant, thereby further contributing to the advantageous properties of the tablet.

The tablet may further comprise at least one further solid form of an API, such as a solid form of a different API, wherein said further solid form of an API is intragranular when the further solid form of an API is a very soluble or freely soluble solid form. The skilled person will appreciate that the concept of keeping very soluble or freely soluble solid forms of an API apart from the disintegrant in the tablet base also applies to the situation where two or more soluble APIs are present in the tablet.

The tablet may be characterized by a hardness of 25-500N, more preferably 50-300N. Hardness being determined according to the method “Resistance to crushing of tablets” (tablet hardness): European Pharmacopoeia, 5.0 2.9.8).

The optionally further pharmaceutically acceptable excipients can be selected from the group consisting of fillers, glidants, and lubricants.

The filler is preferably at most sparingly soluble as determined according to European Pharmacopoeia 9.4, Section 5.11, Character Section in Monograph; more preferably, the filler is microcrystalline cellulose.

The glidants are preferably selected from the group consisting of starch, talcum, colloidal silica i.e. syloid, pyrogenic silica, hydrated sodium silioaluminate, and highly dispersed silicone dioxide (e.g., sold under the trade name “Aerosil®)”; more preferably, the glidant is highly dispersed silicone dioxide (e.g., sold under the trade name “Aerosil®”.

The lubricant is preferably selected from the group consisting of magnesium stearate, calcium stearate, sodium stearate, boric acid, sodium benzoate, sodium oleate, sodium lauryl sulfate (SLS), magnesium lauryl sulfate (MLS), and sodium fumarate; more preferably, the lubricant is magnesium stearate.

The disintegrant may be one or more selected from the group consisting of starch, crosslinked cellulose, crosslinked polyvinylpyrrolidone (PVP), crosslinked alginic acid, and ion exchange resin; preferably the disintegrant is croscarmellose sodium.

The invention further relates to a tablet comprising the rapidly or very rapidly dissolving tablet of the invention, and a coating. The coating may be a gastroresistant coat or a non-gastroresistant coat.

In the tablet and the coated tablet of the invention, the API can preferably be selected from a tetracycline and a GnRH antagonist. A preferred API is a tetracycline such as eravacycline, tigecycline or omadacycline. Another preferred API is a GnRH antagonist such as elagolix. The present invention provides a general concept for the rational design of formulations for a wide variety of active pharmaceutical ingredients. It is possible that somebody had stumbled upon a rapidly or very rapidly dissolving tablet of the present invention in the past accidentally, without understanding the rationale of the present invention. Thus, in one embodiment, the rapidly or very rapidly dissolving tablet comprising a very soluble or freely soluble solid form of an active pharmaceutically ingredient (API) of the present invention does not comprise rapidly or very rapidly dissolving tablets that were approved by a national or regional competent authority to authorize human and/or veterinary medicines for a country and/or a region at the effective date of the present invention, such as the European Medicine Agency, the FDA or the Japanese Pharmaceuticals and Medical Device Agency.

### Methods

### 1. Controlling and Determining the Size Distribution of the Granules

### 1.1 By Sieving

The granulate comprising said very soluble or freely soluble solid form of an API in form of granules to be used in the process of the present invention can be tested for having the desired size distribution as defined herein (e.g., at least 90% w/w of said granules have a size of at least AAAμm) by the European Pharmacopoeia 9.0 method 2.9.38. “Particle size distribution estimation by analytical sieving”. Briefly, sieving is carried out under conditions that do not cause the test sample to gain or lose moisture. The relative humidity of the environment in which the sieving is carried out must be controlled to prevent moisture uptake or loss by the sample. In the absence of evidence to the contrary, analytical test sieving is normally carried out at ambient humidity, e.g. 40% r.h., and at ambient temperature, e.g. at 20° C. A test sample having a mass of 25 g to 100 g is typically used for test sieves having a 200 mm diameter. A dry sieving method with mechanical agitation is employed. Preferred is the apparatus Retsch AS 300 control B with an amplitude setting of 0.4 mm, as employed in the examples of the present invention.

Sieves are selected to cover the entire range of particle sizes present in the test sample, wherein the sieve having a mesh size of 150 μm is the finest sieve at the bottom of a nest of sieves having a recommended square-root 2 progression of the area of the sieve openings. All sieves are weighed beforehand. The sample weight is also determined prior to sieving. The sample is placed on the uppermost sieve of the nest of sieves and sieving is carried out for 5 minutes, when all sieves are weighed. Sieving is continued for 5 more minutes and the sieves are weighed again. Sieving is continued until the mass on all of the test sieves no longer changes by more than 5%. When constant values are achieved all sieves and the collection container below the smallest sieve collecting the fine particles are weighed.

If 90% w/w or more of the tested sample are retained in the sieves having a mesh size of 150 μm or more, then within the meaning of the present invention, a sample consists of “granules, wherein at least 90% w/w of said granules have a size of at least 150 μm”. Preferably more than 90% w/w of the tested sample are retained in the sieves having a mesh size of 150 μm or more, and more preferably more than 95% w/w of the tested sample are retained in the sieves having a mesh size of 150 μm or more. Most preferred, all granules of the tested sample are retained in the sieves having a mesh size of 150 μm or more.

### 1.2 By Microscopy

The microscope images were recorded with a Keyence VHX-5000 microscope (VHX-5000 v1.7.0.4) equipped with a Keyence VHX-5020 camera and a Keyence VHX-ZST objective. The particle selection and calculation of the dimension were done with the integrated software package (VHX-H2M2). The coloured areas, which represent the Elagolix granules, were automatically detected by the software. Thereafter, the detected areas were further optimized by modification of the selection parameters (brightness and color, respectively) to fit the optical impression of the granules. The area of the target object was then measured after shape formatting of the extracted area.

### 2. Analyzing the Surface of the Break of the Tablet

The API (elagolix) was mixed with a dye (indigo blue) in a ratio of 60:40 w/w (tubular mixer, 5 min) and granulated according to the described method (150 bar, 20 mm round punch) and fractionated via sieving. The blue colored granules (>500 μm) were formulated as described (100 bar, 9*19 mm die) and the obtained tablet was analyzed with the digital microscope.

In the case of eravacycline the API was of a yellow colour and the presence of eravacycline-containing granules in the compressed tablets was already evident from visual inspection.

### EXAMPLES

### Example 1 Preparing Tablets Comprising Granules of Different Sizes and Tablet Base

### 1.1 Slugging Procedure:

Amorphous spray dried elagolix sodium was compressed using a single punch tablet press Flexitab S (Röltgen Marking System) using a flat round punch (20 mm diameter) and compaction pressures of 150 bar to 250 bar. To prevent sticking to the die, the punch was brushed with a little magnesium stearate.

Calculation of compaction forces for this punch (20 mm diameter):

| | Area | Compaction pressure | Compaction force |
|---|---|---|---|
| Punch | [mm²] | [bar] | [kN] |
| D20 mm, round | 314 | 150 | 4.71 |
| D20 mm, round | 314 | 200 | 6.28 |
| D20 mm, round | 314 | 250 | 7.85 |

The obtained pellets were carefully broken up and sieved using a sequential sieve set-up to fractionate the granules: 800 μm-500 μm-250 μm-150 μm. This was achieved by starting with a sieve having a 800 μm mesh size and then subsequently using sieves with decreasing mesh sizes corresponding to the indicated values. The sieving apparatus was a Retsch AS 300 control B which was operated with an amplitude of 0.4 mm. Sieving for 5 minutes already provided visibly clean size fractions.

The sieved fractions (800 μm to 500 μm, 500 μm to 250 μm, 250 μm to 150 μm and the smallest fraction <150 μm) were formulated into tablets using following tablet formula:

### 1.2 Tablet Formula:

Granulated elagolix sodium (calculated for 200 mg elagolix=20 wt %)
Croscarmellose sodium (5 wt %)
Magnesium stearate (0.5 wt %)

### Aerosil (0.5 wt %)

Microcrystalline cellulose (up to 100%)

### 1.3 Formulation Procedure:

All extragranular components of the tablet mixtures were sieved previously through an 800 μm or 1000 μm mashed sieve and then thoroughly mixed with the granules using a TURBULA® T2F three-dimensional shaker-mixer (WAB-Group) for 5 min (level 34).

Tablets were pressed on a single punch tablet press Flexitab S (Röltgen Marking System). All tablets were pressed with a 9×19 mm die (lower punch height: 9 mm) using 100 bar compaction pressures (return time: 3 sec).

### 1.4 Dissolution Assay:

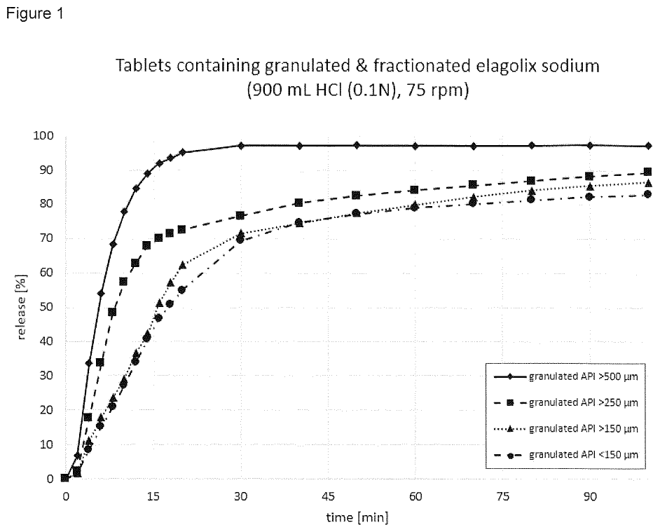

The tablets were then tested in an Agilent dissolution apparatus 708-DS (USP II—Paddles) using 0.1N HCl as medium (900 mL, 75 rpm, detection at 246 nm for elagolix—for later experiments with eravacycline a wavelength of 301 nm was used for detection). It was observed that the tablet containing the largest particles after granulation (500 μm-800 μm sieve fraction, the curve represented by diamonds in FIG. 1) displayed the fastest dissolution profile. The smaller the particles were, the slower the tablet disintegrated and the slower the API dissolved.

### Example 2. Comparison of Different Granulation Pressures

The tablets were prepared as described above in 1, however by applying different granulation pressures: 150 bar, 200 bar, and 250 bar. Elagolix sodium granules corresponding to a size fraction 800 μm>elagolix granules>250 μm were compared to tablets prepared from ungranulated elagolix sodium.

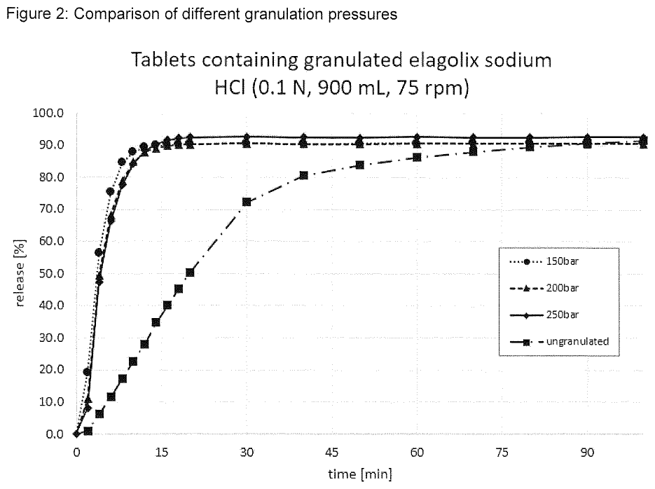

The comparison of the dissolution behavior of the tablets that were prepared by applying different granulation pressures is depicted in FIG. 2.

In this figure, it can be seen that the granulation pressures can vary between 150 bar and 250 bar, without significant difference on the tablets' dissolution behavior.

### Example 3 Tablets Comprising Granulate Comprising Granules of Amorphous Eravacycline Dihydrochloride

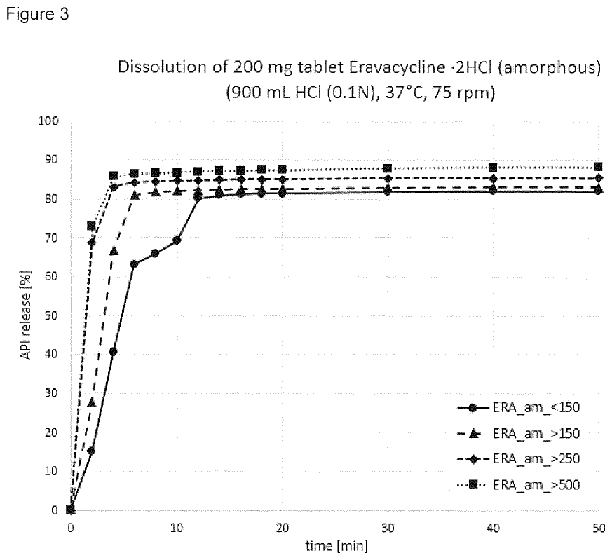

Tablets were prepared essentially as described above in Example 1, but using amorphous eravacycline dihydrochloride (obtainable according to reference) instead of elagolix sodium. Granulate comprising Eravacycline dihydrochloride granules was prepared using a granulation pressure of 150 bar, granules representing different size fractions as indicated were selected—as described in example 1.1 for elagolix—and tablets were prepared—as described in example 1.2 and 1.3 for elagolix. The obtained eravacycline tablets were then tested in the dissolution assay as described above in example 1.4. Tablets prepared from the largest eravacycline granules showed the fastest dissolution (see FIG. 3).

### Example 4 Tablets Comprising Granules of Crystalline Eravacycline Dihydrochloride

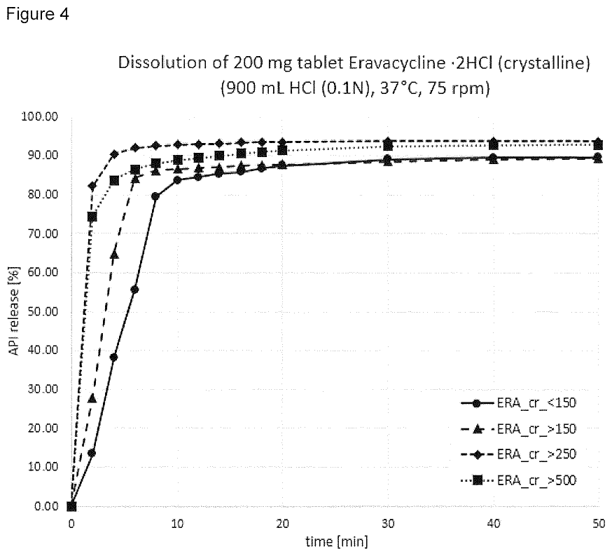

Tablets were prepared and tested as described for example 3, but crystalline eravacycline dihydrochloride replaced amorphous eravacycline dihydrochloride as the API. Tablets prepared from the largest eravacycline granules showed the fastest dissolution (see FIG. 4).

### Example 5 Tablets Comprising Coloured Granules of Elagolix Sodium

Tablets were prepared from elagolix sodium as described in example 1, but during the granulation step methylene blue was added to elagolix sodium so as to allow a visual analysis of the granules-containing tablets after compression. Tablets were broken apart by hand and microscope pictures were taken at a magnification of 20-fold. The microscope images were recorded with a Keyence VHX-5000 microscope (VHX-5000 v1.7.0.4) equipped with a Keyence VHX-5020 camera and a Keyence VHX-ZST objective. The particle selection and calculation of the dimension are done with the integrated software package (VHX-H2M2).

The coloured areas, which represent the Elagolix granules, were automatically detected by the software. Thereafter, the detected areas were further optimized by modification of the selection parameters (brightness and color, respectively) so that the detected areas fit the optical impression of the granules. The areas with the same level (brightness, color or manually selected) were automatically extracted and the area of the target object was measured after shape formatting of the extracted area. Large granules were readily visible in the microscope images, as evident from particles having a large apparent surface area of several tens of thousands of square-micrometers and/or circumferences of several hundred micrometers.

### Example 6 Stress Test of Tablets Comprising Granules of Elagolix Sodium

Tablets were prepared from elagolix sodium as described in example 1 and stress test were performed at 25° C./60% rh (rh=relative humidity) in sealed light- and air tight aluminium compound bags. After one and three months, water content, assay and impurity formation of the stressed tablets were determined.

The water content was measured by coulometric Karl Fischer titration with a Metrohm 832 KF Thermoprep oven and a Metrohm 831 KF Coulometer using a generator electrode without diaphragm and Hydranal-Coulomat AG Oven analyte solution. The sample was heated to 110° C. in the oven and the released water transferred to the titration cell for analysis, using dry air as a carrier gas.

In order to determine the Assay (=recovery of API after storage) and the potential presence of impurities such as lactam degradation products after storage for 0, 1 and 3 months a sample of the API was dissolved in water/acetonitrile mixture (70:30), to obtain a final concentration of 1 mg/mL, and 2.0 μL injected into a YMC Triad C18 (150*4.6 mm, 3 μm, 12 nm) column of an Agilent 1260 Infinity Series equipment with a variable wavelength UV analytical detector at 220 nm. The areas under the peaks of the API and under the peaks of potential impurities/degradation products were analyzed using the software Agilent ChemStation and calculated as percentage of the starting material compared to a reference solution with known concentration. Flow was 1.2 mL/min., temperature was 20° C., Eluent A was Sulfamic acid (10 mM; pH=2.0) and Eluent B was acetonitrile. Eluent profile was: at 0 min. 30% Eluent B, at 10 min. 95% Eluent B, at 15 min 95% Eluent B and at 15.1 min. 30% Eluent B.

25° C./60% rh

| Analysis | native | 1 month | 3 months |
|---|---|---|---|
| Water content [%] | 3.56 | 3.55 | 4.05 |
| Assay [%] | 91.4 | 106.8 | 99.4 |
| Impurity formation | Not detectable | Not detectable | Not detectable |
| (lactam) | | | |

No lactam formation or degradation of the API could be observed after 3 months at 25° C. and 60% rh.

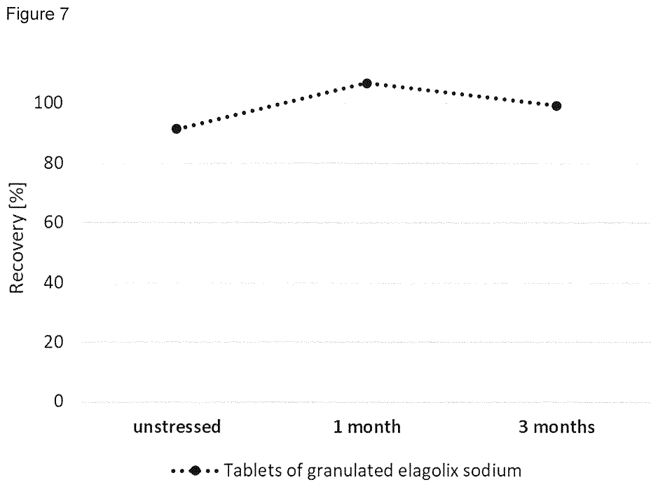

FIG. 7 shows the recovery of Elagolix after stress test of tablets comprising granules of elagolix sodium. The x-axis denotes duration of stress test, the y-axis the percentage of recovered Elagolix at a given time point.

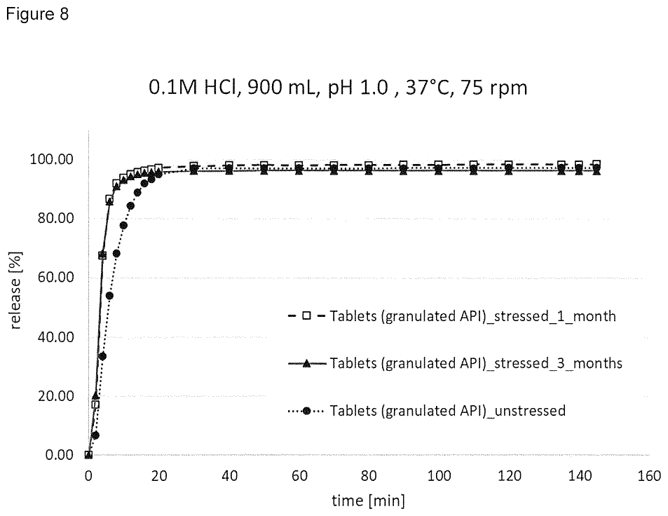

FIG. 8 shows the dissolution profiles of tablets containing compressed pure granulated and fractionated elagolix sodium (the API) after stress tests at 25° C./60% rh. The x-axis denotes time in minutes, the y-axis the percentage of dissolved API at a given point in time. The release (dissolution)-profiles were obtained as described in the examples. No change in the dissolution kinetic was observed after the stress test.

1. Process for the preparation of a rapidly or very rapidly dissolving tablet comprising a very soluble or freely soluble solid form of an active pharmaceutically ingredient (API), wherein the tablet comprises a granulate and a tablet base, wherein the process comprises the following steps:
a) preparing said granulate comprising said very soluble or freely soluble solid form of an API in form of granules, wherein at least 90% w/w of said granules have a size of at least 150 μm as determined by sieving, wherein
(i) the solid form of the API is very soluble or freely soluble in FASSIF-medium according to European Pharmacopoeia 9.4, Section 5.11, Character Section in Monograph; or
ii) the solid form of the API is very soluble or freely soluble in 0.1N HCl-solution according to European Pharmacopoeia 9.4, Section 5.11, Character Section in Monograph;
b) mixing the granulate obtained in step a) with said tablet base comprising one or more disintegrants and optionally further excipients;
c) compressing the mixture from step b) to form said tablet,
wherein said granulate comprises at least 90% w/w of the total amount of said very soluble or freely soluble solid form of the API being present in the tablet,
wherein the amount of said very soluble or freely soluble solid form of said API in the tablet is at least 10% w/w based on the weight of the tablet,
wherein the tablet base comprises one or more disintegrants, and
wherein the amount of said very soluble or freely soluble solid form of an API in said granulate of step a) is at least 50% w/w, based on the total weight of said granulate.

2. The process according to claim 1, wherein the granules of step (a) have a size of at least 200 μm.

3. The process according to claim 1, wherein the amount of said very soluble or freely soluble solid form of said API in the tablet is from 15% w/w to 60% w/w based on the weight of the tablet.

4. (canceled)

5. The process according to claim 1, wherein the granulate is prepared by a dry granulation process.

6. The process according to claim 1, wherein the amount of said very soluble or freely soluble solid form of said API in the granulate of step a) is at least 90% w/w, based on the total amount of API being present in the tablet.

7. The process according to claim 1, wherein the tablet base comprises at least 90% w/w of the total amount of disintegrant being present in the tablet.

8. Process for the preparation of a coated tablet comprising the preparation of a tablet according to claim 1, further comprising a step of coating the tablets.

9. The process according to claim 8, wherein the coating is a gastroresistant coat and the the solid form of the API is very soluble or freely soluble in FASSIF-medium according to European Pharmacopoeia 9.4, Section 5.11, Character Section in Monograph.

10. (canceled)

11. Compressed rapidly or very rapidly dissolving tablet comprising a very soluble or freely soluble solid form of an active pharmaceutically ingredient (API), wherein the tablet comprises a mixture of a granulate and a tablet base, wherein
the granulate comprises a very soluble or freely soluble solid form of an active pharmaceutically ingredient (API) in form of granules, wherein
(i) the solid form of the API is very soluble or freely soluble in FASSIF-medium according to European Pharmacopoeia 9.4, Section 5.11, Character Section in Monograph; or
(ii) the solid form of the API is very soluble or freely soluble in 0.1N HCl-solution according to European Pharmacopoeia 9.4, Section 5.11, Character Section in Monograph;
wherein said granulate comprises at least 90% w/w of the total amount of said very soluble or freely soluble solid form of the API being present in the tablet, and wherein the amount of said very soluble or freely soluble solid form of an API in said granules is at least 50% w/w, based on the total weight of said granules; and
the tablet base comprises one or more disintegrants and optionally further excipients, wherein the amount of said very soluble or freely soluble solid form of said API in the tablet is at least 10% w/w based on the weight of the tablet and wherein, when the tablet is broken in half by hand and the surface of the break is analyzed, at least five of said granules have an apparent surface area of at least 30000 μm².

12. The tablet according to claim 11, wherein the amount of said very soluble or freely soluble solid form of said API in the tablet is from 15% w/w to 60% w/w, based on the total weight of the tablet.

13. (canceled)

14. Compressed rapidly or very rapidly dissolving tablet comprising a very soluble or freely soluble solid form of an active pharmaceutically ingredient (API), wherein the tablet comprises a mixture of a granulate and a tablet base, wherein the compressed tablet is obtainable from a process according to claim 1.

15. Coated tablet comprising the rapidly or very rapidly dissolving tablet according to claim 11.
